(19) United States
(12) Patent Application Publication
(10) Pub. No.: US 2016/0124405 A1
(43) Pub. Date: May 5, 2016

(54) THERMAL OPTIMIZATION OF AN IMAGING SCAN ROOM

(71) Applicant: GENERAL ELECTRIC COMPANY, SCHENECTADY, NY (US)

(72) Inventor: Gary Dwayne Mandrusiak, Latham, NY (US)

(21) Appl. No.: 14/530,327

(22) Filed: Oct. 31, 2014

(51) Int. Cl. G05B 17/02; G06F 17/50; G06K 9/20; G06F 3/0486; G06F 3/0481; G06F 3/0484; A61B 19/00; G06F 17/16

(52) U.S. Cl. CPC G05B 17/02; A61B 19/5225; G06F 17/5009; G06F 17/16; G06F 3/0486; G06F 3/0481; G06F 3/04842; G06K 9/2054; A61B 2019/5236; A61B 2019/5242; A61B 2019/5276

(57) ABSTRACT

In accordance with the present disclosure, a tool for evaluating the thermal layout of a scan room is discussed. In practice, the tool may be used to quickly generate and test different room and imaging system layouts to identify a suitable layout. A scan room and imaging system may then be placed and oriented in accordance with the layout that has been tested and found acceptable using the tool.

### BACKGROUND

Embodiments of the invention generally relate to approaches for managing the thermal environment in a room or facility in which imaging devices are employed.

In a medical context, imaging systems (such as general radiography X-ray system, tomosynthesis system, computed tomography (CT) system, mammography system, C-arm angiography system, single photon emission computed tomography (SPECT) system, positron emission tomography (PET) system, ultrasonic imaging system, magnetic resonance imaging (MRI) system, nuclear medicine imaging system, and various other modalities) create images of a patient or object using a variety of physical principles. For example, certain such physical principles may relate to the differential transmission of radiation (e.g., X-rays) through the body, the transmission of acoustic waves through the body, the paramagnetic properties of the body, or the localization of and breakdown of radiopharmaceuticals in the body. Images generated using these various principles usually provide additional insight into structural or functional features of a patient without the need for invasive procedures. As such, these approaches are a valuable tool for diagnosticians and researchers who wish to non-invasively gain additional information about the internal structures or functioning of a patient.

In many instances, the imaging devices or systems are housed in dedicated rooms within a facility, such as a hospital. In general, the layout and size of these dedicated rooms is not uniform and may in fact vary considerably both within and between facilities. As a result, the control of the thermal environment in such rooms may be difficult to standardize to the differing types of rooms in which imaging systems may be housed.

By way of example, the operating requirements for a given type of imaging system may specify that the air temperature within the room, or at certain locations of the imaging system, should be kept within a certain range. However, due to the layout or size of this room, these requirements may be difficult to meet when the imaging system is in operation. Alternatively, the measures needed to meet the thermal requirements for the imaging system within a given room may result in an uncomfortable environment for patients within the room during an examination. Thus, situations may arise where the imaging system is properly cooled but the patient is uncomfortable (e.g., cold), the patient is comfortable but the imaging system is improperly cooled, or, in the worst case, the imaging system is improperly cooled and the patient is uncomfortable.

### BRIEF DESCRIPTION

In one embodiment, a computer-implemented method for modeling patient comfort in a scan room is provided. In accordance with this method an act is performed of receiving, on a processor-based system, a set of inputs comprising: dimensions and shape of the scan room, placement of one or more imaging system components in the scan room, and placement and operational characteristics of air supplies within the scan room. A cellular mesh is generated based on the set of inputs. An air flow vector is computed for each cell of the cellular mesh based on the set of inputs and a set of boundary conditions. A temperature is computed for each cell of the cellular mesh using the computed air flow vectors and a set of boundary conditions. A measure of projected patient comfort is computed based at least on the computed temperatures, velocities, and the set of inputs. The measure of projected patient comfort is displayed as a factor in evaluating layout of the scan room described by the set of inputs.

In another embodiment, a computer-implemented method for modeling patient comfort in a scan room is provided. In accordance with this method an act is performed of receiving, on a processor-based system, a set of inputs comprising: dimensions and shape of the scan room, placement of one or more imaging system components in the scan room, and placement and operational characteristics of air supplies within the scan room. A cellular mesh is generated based on the set of inputs. An air flow vector is computed for each cell of the cellular mesh based on the set of inputs and a set of boundary conditions. A temperature is computed for each cell of the cellular mesh using the computed air flow vectors. A determination is made whether one or more boundary temperatures are converged. If the one or more boundary temperatures are determined to not be converged, the set of boundary temperatures are updated until the boundary temperatures are determined to be converged. A set of outputs are displayed comprising one or more of estimated temperatures of all or part of the scan room or estimated temperatures of all or part of the imaging system components.

In a further embodiment, a graphical user interface is provided. The interface includes: a plurality of input fields, each field corresponding to a parameter defining a patient procedure room or a system to be deployed in the patient procedure room; a layout pane configured to allow drag-and-drop placement and orientation of the system within the patient procedure room; a plurality of room temperature fields, each room temperature field corresponding to estimated temperature for all or a portion of the patient procedure room; a plurality of system temperature fields, each system temperature field corresponding to estimated temperature for a system component; and one or more patient comfort index fields, each patient comfort index field corresponding to an estimated patient comfort.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a pictorial view of a CT imaging system adapted for generating images in accordance with aspects of the present disclosure;

FIG. 2 is a schematic block diagram of the exemplary imaging system illustrated in FIG. 1;

FIG. 3 depicts an overhead view of a scan room and installed imaging system, in accordance with aspects of the present disclosure;

FIG. 4 depicts a process flow diagram of an algorithm for implementing the present modeling tool, in accordance with aspects of the present disclosure;

FIG. 5 depicts an overhead plan view of a modeled scan room showing aspects of a potential flow approach for estimating air flow, in accordance with aspects of the present disclosure;

FIG. 6 depicts an overhead plan view of a modeled scan room showing aspects of a temperature estimation approach, in accordance with aspects of the present disclosure;

FIG. 7 depicts an example of an input screen of a GUI for implementing the present tool, in accordance with aspects of the present disclosure;

FIG. 8 depicts an alternative example of an input screen of a GUI for implementing the present tool, in accordance with aspects of the present disclosure;

FIG. 9 depicts an example of an output screen of a GUI for implementing the present tool, in accordance with aspects of the present disclosure; and

FIG. 10 depicts an alternative example of an output screen of a GUI for implementing the present tool, in accordance with aspects of the present disclosure.

### DETAILED DESCRIPTION

The present disclosure relates approaches for modeling environmental conditions within an environment in which an imaging system is deployed. In this approach, different sizes and layouts of rooms may be modeled as well as different types of imaging system and environmental and ventilation schemes. A user may model different position and orientation of the imaging system within the room as well as different placement and usage of ventilation and temperature control within the room. Based on the modeling, the user may generate a suitable temperature control scheme and implement such a scheme so as to adequately cool the imaging system within the room while providing a comfortable patient environment. For example, in certain implementations a detailed three-dimensional representation of the thermal and system performance consequences of a proposed system and room layout may be generated for evaluation by a user.

As will be appreciated, the present approach may be suitable for use in planning an environment for various types of imaging systems. For the purpose of illustration and to facilitate explanation, various examples may be discussed herein related to specific types of imaging systems, such as CT imaging systems. It should be appreciated, however, that such examples are provided merely to simplify explanation. It should be understood that the present approach may also be suitable for use in planning environments for other types of imaging systems, including, but not limited to, general radiography X-ray systems, tomosynthesis systems, mammography systems, C-arm angiography systems, SPECT systems, PET systems, ultrasound systems, nuclear medicine imaging systems, and MRI systems. In addition, though the present examples are generally directed to imaging systems, the present approach may be suitable for use in other medical contexts, such as for modeling or planning layouts for operating or interventional rooms which may or may not include imaging equipment as described herein.

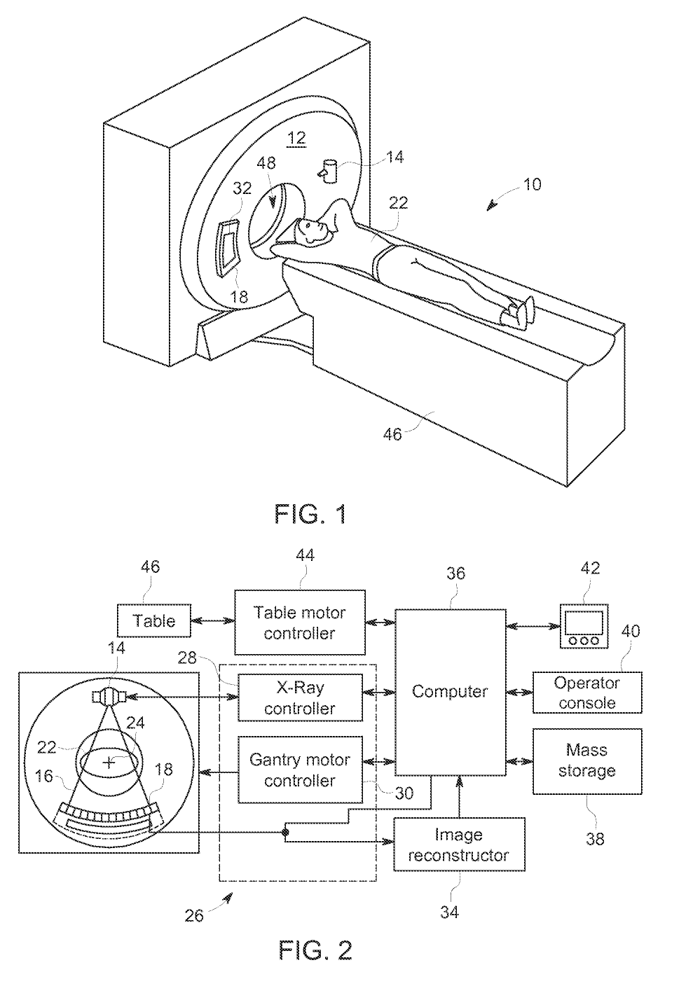

With the preceding in mind, and turning to FIG. 1, an example of a CT imaging system 10 is shown to help visualize the footprint and physical constraints imposed by such a system. As will be appreciated, other types of imaging modalities may have a comparable footprint in terms of spatial requirements and in terms of cooling needs. As depicted in FIGS. 1 and 2, the CT imaging system 10 includes a gantry 12 that has a radiation source 14 (e.g., an X-ray tube) that projects X-rays 16 toward a detector assembly 18 on the opposite side of the gantry 12.

Referring now to FIGS. 1 and 2, detector array 18 is formed by a plurality of detector elements and a data acquisition system (DAS). The plurality of detector elements sense the incident X-rays that pass through a patient 22 positioned between assembly 18 and source 14. During a scan to acquire X-ray projection data, the gantry 12 and the components mounted thereon rotate about a center of rotation 24.

Rotation of gantry 12 and the operation of X-ray source 14 are governed by a control mechanism 26 of CT system 10. The control mechanism 26 may include an X-ray controller 28 that provides power and timing signals to an X-ray source 14 and a gantry motor controller 30 that controls the rotational speed and position of gantry 12. An image reconstructor 34 receives sampled and digitized X-ray data from DAS and performs image reconstruction. The reconstructed image is applied as an input to a computer 36, which stores the image in a mass storage device 38 and/or displays the image on a display 42. The associated mass storage device 38 may store programs and codes executed by the computer, configuration parameters, and so forth. The computer 36 may also receive commands and scanning parameters from an operator via console 40 that has some form of operator interface, such as a keyboard, mouse, voice activated controller, or any other suitable input apparatus. In addition, computer 36 operates a table motor controller 44, which controls a motorized table 46 to position patient 22 and gantry 12. Particularly, table 46 may be configured to move the patient 22 through a gantry opening 48 during operation of the CT system 10.

With the preceding discussion in mind, it should be appreciated that the CT system 10 may generate heat during operation due to the operation of the electrical components and/or due to the operation of the radiation generating source 14. Thus, during operation, the CT system 10 may require cooling in the general operating environment, and in some instances, may benefit from more direct or applied cooling or ventilation at specific locations of the system 10, such as at locations where heat is generated and/or at locations where air inlets are provided to help circulate air around or in the system 10. As will also be appreciated, the size and shape of the CT system 10 may in practice affect the flow of air in the room in which the system 10 is located in general or, in the localized environment around the system 10 in particular. While a CT imaging system 10 has been depicted and described, it should be understood that these came considerations are equally applicable in other imaging contexts and for other types of imaging systems.

In practice imaging systems such as the CT imaging system 10 described above may be designed with various thermal conditions and expectations in mind Guidance may be provided to a facility operator or installer to meet these thermal constraints when placing an imaging system in an examination room. For example, for a given imaging system, such as a CT imaging system 10, room heat generation rates, room temperature ranges, and recommended placements for heating, ventilation, and air conditioning (HVAC) air supplies and returns may be provided with the system to facilitate installation.

While this information may be used to set air supply flow rates and air temperatures for air supplied to the scan room, the temperature requirements for the imaging system may still not be met due to the size and/or layout of the scan room, potentially resulting in scanner overheat alarms and downtime. In particular, the rooms in which such systems are placed may vary significantly in size, layout, ventilation, and so forth, both within a facility and between facilities, making standardized recommendations inadequate in some instances. Further, attempts to meet the specified thermal requirements for the imaging system may create situations where the thermal requirements for the system are met, but only at the expense of patient comfort. For example, positioning the system 10 so that cold air blowing on to the system provides sufficient cooling may result in the cold air also blowing on the patients undergoing imaging. In a worst case scenario, the system 10 may not be adequately cooled while still creating patient discomfort.

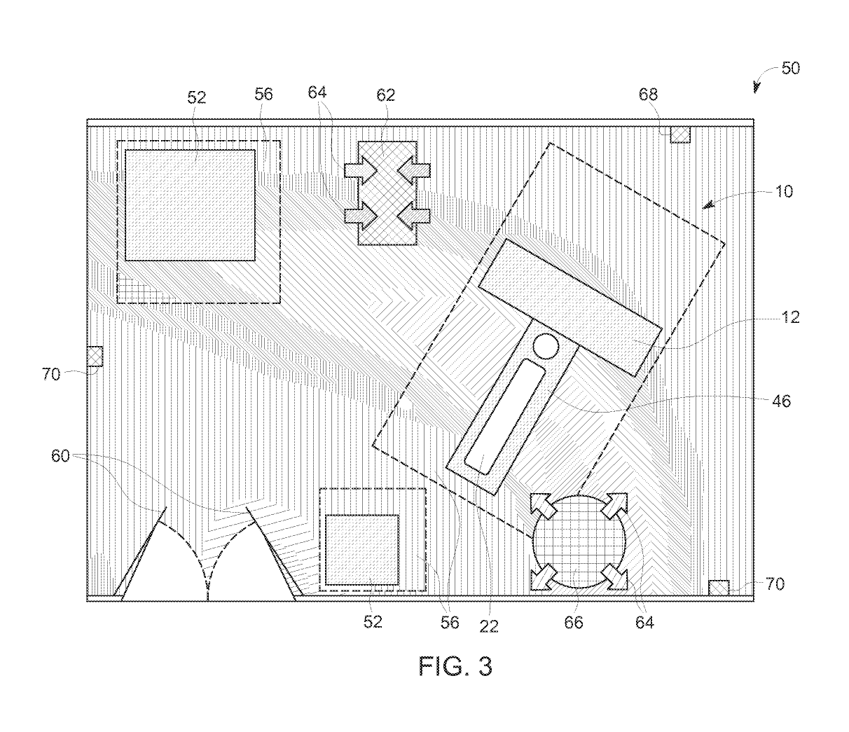

For example, turning to FIG. 3, an example scan room 50 is depicted from an overhead or plan perspective. As noted above, though imaging systems and scan rooms are generally discussed herein as suitable rooms to model and to facilitate explanation, it should also be appreciated that other types of medical facilities, including operating and interventional rooms, may also benefit from the presently described approaches and tools. In the depicted scan room 50, a CT system 10 is depicted which has a gantry 12 and table 46 on which a patient 22 may be supported. Other components of the CT system 10 include operating cabinets 52, which may include various operational or computational components of the CT system 10, such as the operator console 40, display 42, storage 38, and/or computer 36, as well as the various controllers discussed herein. For example, one cabinet may contain computational and/or storage components, while another cabinet is devoted largely to providing an operator interface or console.

In the depicted example, each of the CT system components has a corresponding workflow clearance or envelope 56 (denoted by dashed lines) that encompasses an area or volume that is utilized to operate or service the component. For example, the workflow clearance 56 defined for the gantry 12 and table 46 may encompass that area or volume needed to accommodate rotation of the gantry 12, movement of the table 46, electrical and data connections, patient and operator access and so forth. Likewise, the cabinets 52 may define respective workflow clearances 56 that provide space for operator movement or access, such as being able to open and close access panels or to operate with interface devices on the cabinets 52.

Each workflow clearance 56 may also be partly determined by various air inlets and outlets of the respective components of the CT system 10 that provide circulation of cooling air with respect to the components. For example, with respect to the gantry 12, air inlets may be provided on the sides of the gantry 12 that allow air from the surrounding environment to be drawn into the gantry 12, circulated to absorb operational heat, and expelled through air outlets on the top of the gantry 12 to dissipate heat from the gantry 12 during operation. Similarly, the computational and operational components in the cabinets 52 may also be cooled using air drawn into the cabinets through inlets, which is then circulated within the respective cabinet to dissipate heat, and expelled through air outlets provided in the cabinets. As will be appreciated to work properly, such air inlets and outlets may require open space provided near the inlet or outlet in order for air to sufficiently circulate to provide the desired degree of cooling and heat dissipation. This space may also be accounted for in the respective workflow clearances 56.

Thus, the spatial requirements of each component of the CT system 10 are not defined solely by the physical space occupied by a given component at a given time, but also by the surrounding space needed to actually operate, access, and cool the component. The respective workflow clearances 56 of a given component may, therefore, limit the possible placement and arrangement of the components of the CT system 10 within the scan room 50.

Further, the scan room 50 itself likely imposes certain constraints on the placement and positioning of the CT system 10 within the scan room. For example, in the depicted scan room 50, a pair of doors 60 may open into the scan room 50 and may require a certain clearance be provided. Likewise, electrical and/or plumbing connections within the scan room 50 may impose constraints on placement and positioning of the components of the CT system 10.

In the depicted example heating, ventilation, and air conditioning features of the scan room 50 are also depicted. Such features determine the flow and temperature of air circulating in the scan room 50 and are generally used to provide the needed cooling to the scan room 50. For example, in FIG. 3 the placement of an air return 62 is depicted into which warm or environmental air 64 is pulled as part of the climate control for scan room 50. In general, the air return 62 may be located on a wall or in the ceiling of the scan room 50. Similarly, an air supply 66 is shown through which conditioned (e.g., cooled or heated) air 68 is expelled into the scan room 50 to achieve maintain the scan room 50 at a specified temperature. As will be appreciated, though only a single air return 62 and air supply 66 are depicted in FIG. 3, in practice a scan room 50 may contain multiple air returns and/or supplies. The temperature setting for the scan room 50 (and, thus, the activity of the air returns 62 and air supplies 66) may be controlled by a thermostat 68 provided in the scan room 50 or in a nearby room. In addition, control signals or temperature data may be provided to the thermostat 68 or to other controllers of the HVAC system via one or more additional temperature sensors 70 separate from the thermostat 68 that may also be provided in the scan room 50. Taken together, the respective imaging system components and their placement, the room size and layout, the room fixtures, and the respective HVAC system within the scan room 50 determine the flow and temperature of air within the scan room 50.

As noted above, installation and operation guidelines provided with a given imaging system (such as CT system 10 of this example) typically specify temperature recommendations and limits (i.e., minimums and maximums) for the scan room 50 and, possibly, for individual components of the CT system 10 (such as temperatures at the air inlets and outlets for such components). As noted above, in practice it may be difficult to achieve the specified temperature ranges while maintaining a comfortable patient environment. In particular, scan room specific considerations, such as those described with respect to FIG. 3, may make it difficult, for a given scan room 50 to be maintained within the specified temperature ranges. Further due to the size and sensitivity of the equipment in question, it is generally not feasible to repeatedly or continually rearrange equipment to determine the effects on the environmental thermals.

With this in mind, the present approach provides a software-based engineering tool suitable for rapidly evaluating the thermal layout of a hospital scan room. This tool may be used by an operator to specify different placements of an imaging system within a given scan room 50 and to evaluate the thermal consequences of such different placements of the imaging system components. In certain embodiments the tool allows for arbitrary placement of imaging system hardware (for any imaging system modality) and models the resulting three-dimensional (3D) temperature profile and air velocities. Results may be provided in color or gray-scale to facilitate review and understanding of the results. Further, the tool may execute a modeling run in under a minute. The tool may also allow for the modeling, explicitly or implicitly, of patient thermal comfort. In this manner, the tool provides visual output describing the thermal consequences of a particular layout of an imaging system within a given scan room 50, including the consequences of a poor layout.

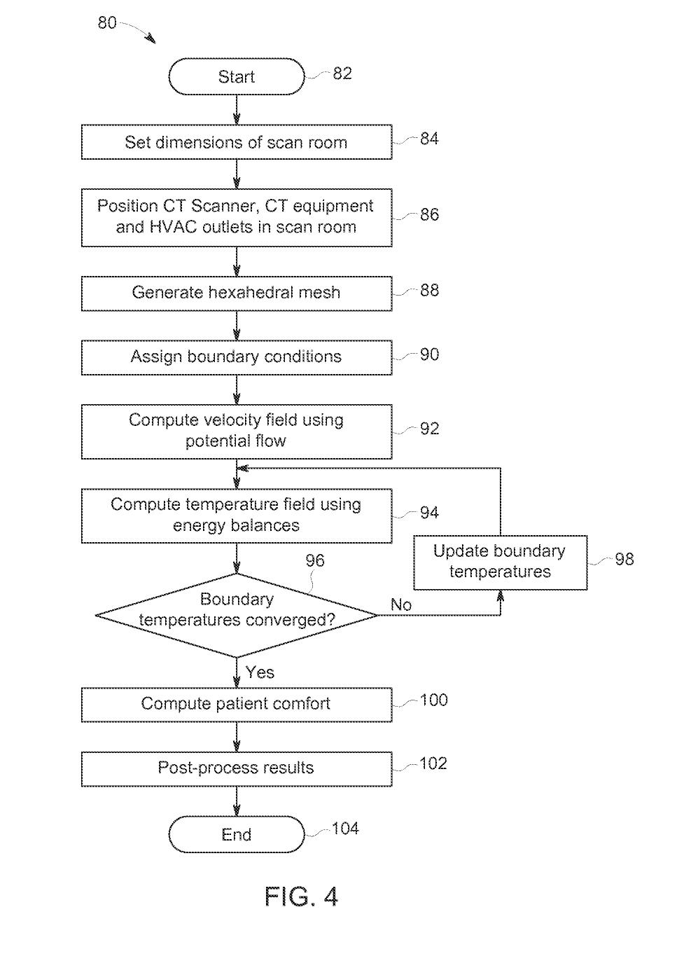

With this in mind, and turning to FIG. 4, a process flow diagram 80 is depicted describing various steps that may be implemented by a software tool as described herein. At the beginning (block 82) of the depicted process flow, a user may specify (block 84) the dimensions of the scan room 50. Thus, at this step the user may specify the physical layout of the scan room 50, including the size and shape of the scan room 50 as well as the presence and dimensions of physical features of the scan room 50 (such as doors 60, partitions, counters, and so forth).

Once the physical layout of the scan room 50 is established in the tool, the user may then position (block 86) the imaging system components (e.g., the CT scanner, the CT computational and control equipment, the CT power supply, and so forth) within the modeled scan room. In addition, at this stage, the user may position the HVAC features (e.g., the air supplies 66 and the air returns 64) within the room as well as any thermostats 68 or temperature sensors 70. The use may also specify the HVAC flow rate and temperature information (e.g., temperature at the air supplies) at this step. Consequently, the energy costs associated with the modeled layout may also be determined based on these HVAC stipulations. Thus, energy efficiency, as derived based on the specified HVAC flow rates and temperatures, may also be assessed in addition to air flow velocity, room and equipment temperature, and patient comfort, which are each discussed in greater detail below.

Based upon these input scan room features and dimensions, a modeling algorithm employing potential flow theory is employed to generate a reduced-physics estimate of the three-dimensional air velocity and temperature fields associated with the proposed room and system layout. In particular, implementations of the potential flow theory calculations and computations may assume no viscosity, irrotational flow, and/or other physics-simplifying assumptions (in contrast to computational fluid dynamics approaches) to generate estimates of velocity field in the modeling process. The velocity field computed in this manner may then be used in a companion energy balance model that computes the corresponding temperatures at each cell to generate temperatures fields or profiles for the modeled layout. In practice, each reduced physics estimate may be generated in under a minute (e.g., 30 second, 20, seconds, 10, second or less), in contrast to modeling processes that do not use reduced-physics modeling techniques, such as computational fluid dynamics techniques.

For example, in one implementation, as part of the potential flow theory estimation, a representative cellular mesh (e.g., a hexahedral computational mesh), is generated (block 88) to facilitate and simplify modeling of the thermal profile of the scan room under different scenarios. This computational grid is readjusted whenever changes are made to the modeled room or layout, such as to try alternative layouts or scenarios in subsequent modeling runs. In this example, air flow and temperature calculations (as discussed below) are performed on the current hexahedral computational mesh generated for the input scan room layout and imaging system placement. In the present example where potential flow theory is employed, mesh points are identified that correspond to or are occupied by solid objects (e.g., walls, the CT gantry, the CT table, CT system cabinets) and these mesh points are blocked out of the calculations. In addition, in certain embodiments, mesh points defining or adjacent to boundary regions (e.g., domain boundaries or walls, air inlets, outlets, returns or supplies) are identified for appropriate handling in subsequent calculations.

In addition, in the depicted example, the user or system may assign (block 90) one or more boundary conditions, such as limit conditions to be observed at the air inlets and outlets of the imaging system components and/or HVAC supplies and returns. For example, a user may specify the temperatures, or at least the initial temperatures for a scenario, observed at the interface regions associated with the inlets, outlets, supplies, and returns. In certain implementations, such boundary conditions may be supplemented or iteratively updated based upon temperature data modeled at one or more control points (e.g., thermostat 68 or temperature sensors 70).

In the depicted example, the air velocity field in the modeled domain is computed (block 92) using potential flow. As noted above, the velocity field may be computed using potential flow theory techniques that employ assumptions such as no viscosity and irrotational flow to simplify the physics model. In this manner, the computational requirements and time may be reduced to allow rapid velocity field modeling of a given room and system layout.

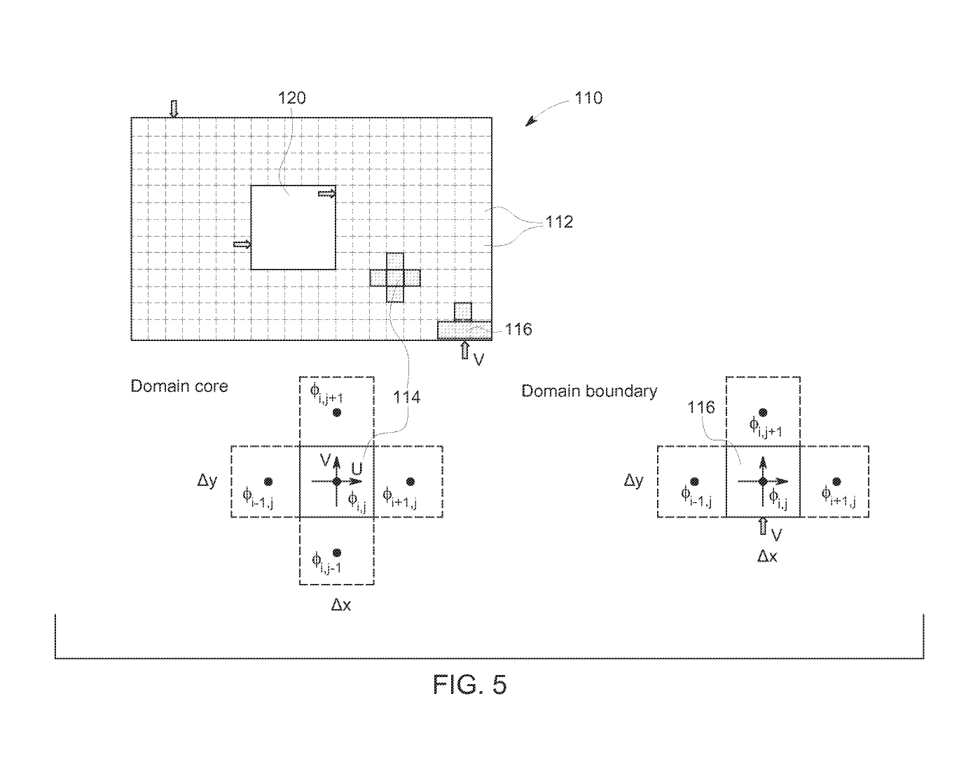

Turning briefly to FIG. 5, an example of one such potential flow approach is graphically depicted. To facilitate explanation of this approach, this discussion will review the methodology using geometry and equations for a two-dimensional layout. In practice the implementation would be three-dimensional, with the governing equations and numerics extended accordingly. In this example, a gridded plan view 110 of a scan room 50 is provided which is broken into cell components 112. Close-up views of two cells, a core cell 114 within the modeled domain and a boundary cell 116 within the modeled domain are provided. As will be appreciated, the core cell 114 has adjacent cells in four directions that can affect air flow in the cell at issue while the boundary cell 116, by virtue of being on the domain boundary has only three adjacent cells that can affect air flow within the modeled cell. In addition, a solid cabinet 120 is also depicted as being present in the room. Various flows of air, such as into and out of the cabinet 120 or into the scan room are depicted via arrows with respect to the plan view.

As noted above, in a potential flow theory implementation, as described herein, fluid is considered inviscid and irrotational to simplify the physics of the system. Air flow velocity is provided by a potential, φ, such that:

$$\begin{matrix}U = \frac{\partial φ}{\partial x} and & ( 1 ) \\ V = \frac{\partial φ}{\partial y} . & ( 2 )\end{matrix}$$

With these expressions, conservation of mass becomes a Laplacian, giving:

$$\frac{\partial U}{\partial x} + \frac{\partial V}{\partial y} = 0 ⇒ \frac{{\partial }^{2} φ}{\partial {x}^{2}} + \frac{{\partial }^{2} φ}{\partial {y}^{2}} = 0. \qquad ( 3 )$$

At boundaries, the velocities U or V in Equations (1) or (2) would be a known flow velocity specific to the HVAC vent or component flow vent as appropriate. In this manner, based on potential flow theory, an air flow vector may be computed for each modeled cell within the domain.

Turning back to FIG. 4, in a given scenario, the temperature field for the modeled layout may be computed (block 94) using the air velocity field generated using potential field theory. For example, in one implementation, the velocity field is used by an energy balance model to compute the corresponding temperatures at each modeled cell corresponding to the proposed layout. For example, temperatures at each cell may be derived from enthalpy transport with the energy balance giving the temperatures and heat loads giving component AT. Iterations of this modeling process may be performed to adjust the boundary temperatures as needed.

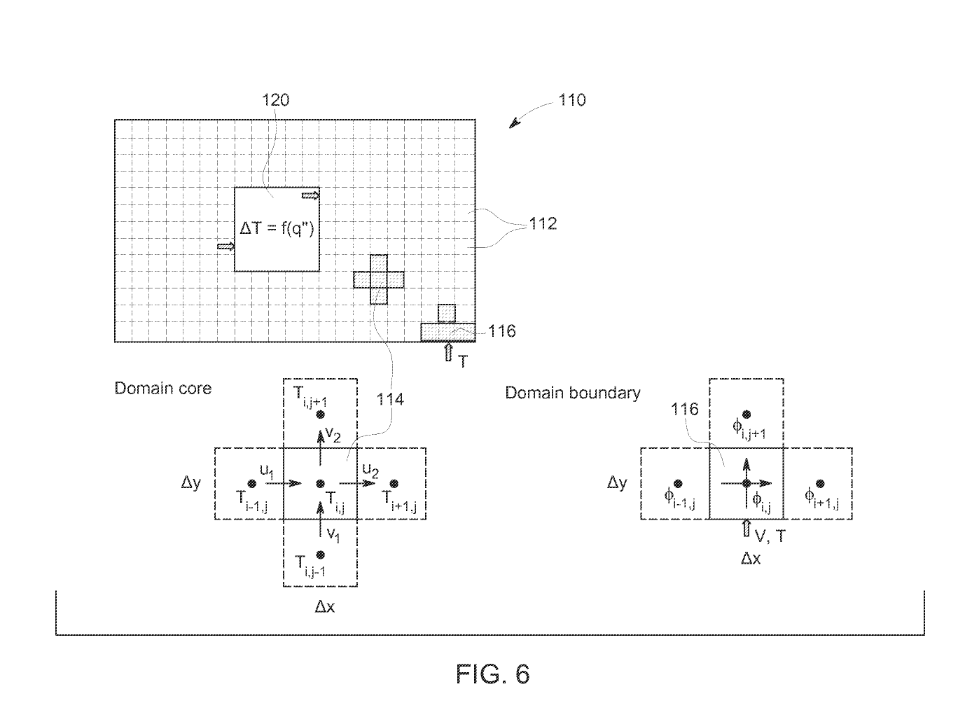

Turning to FIG. 6, an example of temperature filed computation approach is graphically depicted. To facilitate explanation of the present approach, this discussion will review the methodology using geometry and equations for a two-dimensional layout. In practice the implementation would be three-dimensional with the governing equations and numerics extended accordingly. In this example, the gridded plan view 110 of a scan room 50 from FIG. 5 is used again for temperature computations. Close-up views of two cells, the core cell 114 within the modeled domain and the boundary cell 116 within the modeled domain are provided. As in the preceding example, the core cell 114 has adjacent cells in four directions that can affect air flow and temperature within the cell at issue while the boundary cell 116, by virtue of being on the domain boundary has only three adjacent cells that can affect air flow and temperature within the modeled cell. In addition, a solid cabinet 120 is also depicted as being present in the room. Various flows of air, such as into and out of the cabinet 120 or into the scan room are depicted via arrows with respect to the plan view, with cold air flowing into the room and cabinet 120 and heated air flowing out of the cabinet 120.

As noted above, in this example temperature at each cell 112 is derived using an energy balance approach and assuming constant fluid properties. With respect to the core cell 114, the velocity is obtained from Equations (1) and (2) after solving for the potential from Equation (3). These velocities are then used in an energy balance to obtain the temperature at the cell in question using:

$$ρ {C}_{p} U \frac{\partial T}{\partial x} + ρ {C}_{p} V \frac{\partial T}{\partial y} = ∇ \cdot ( k ∇ T ) + q . \qquad ( 4 )$$

The first term on the right hand side of Equation (4) represents turbulent and/or diffusion transport of thermal energy, which the current approach assumes negligible compared to the advective transport represented by the left hand side. The quantity q represents a local heat load. Boundary temperatures are applied by assigning specific values to the appropriate computational cells when solving Equation (4). In this manner, an energy profile or representation of the modeled layout may be generated depicting the estimated temperature at each cell of the modeled scan room.

Turning back to FIG. 4, in this example a determination (block 96) is then made as to whether the boundary temperatures are converged. In practice, such a determination may be made by comparing boundary conditions between successive loops. If the boundary conditions are within some threshold value to one another (such as between 1° C. or 0.5° C.) between iterations, the boundary temperatures may be considered converged. If a determination is made that the boundary temperatures are not converged, the boundary temperatures may be updated (block 98) one or more times (e.g., iteratively), until a determination is made that the boundary temperatures are converged.

Once boundary temperatures are determined to be converged, an estimation of patient comfort for the proposed layout may be computed (block 100). Patient comfort may be estimated based on the estimated temperatures and air flow velocities near the patient table. For example, air velocity (i.e., flow) and temperature calculations may be used as inputs to a human thermal comfort model (e.g., the Fanger or Zhang models) to translate air velocity and temperature information into an estimated patient comfort (e.g., a quantitative or qualitative patient comfort index or measure).

By way of example, one such patient thermal comfort model may distinguish between thermal sensation and thermal comfort of the patient, with the air flow and temperature calculations described above, in conjunction with modeled parameters of the physiology of the patient, being used to derive both the overall thermal sensation observed by the patient and the overall thermal comfort of the patient. Such an assessment may be presented in either a qualitative or quantitative manner for review by the user.

In other modeling approaches, the air flow and temperature calculations described herein may be used to model radiative and convective thermal exchanges by the patient with the environment. These terms, in conjunction with other factors such as mechanical work, metabolic heat production, wet and dry heat exchanges in respiration, evaporation losses due to sweating, and conduction to or from clothing, may be used to assess patient thermal comfort in a qualitative or quantitative manner.

The model results may then undergo post-processing (block 102) and the process is ended (block 104) for the layout under investigation. An output of the depicted process, as discussed herein, may be a detailed, three-dimensional representation (e.g., a two- or three-dimensional image) describing the air flow, thermal and system performance consequences of the modeled scan room and imaging system layout. Because of the use of a reduced-physics estimation procedure, a user can alter the proposed room or system layout based on the obtained results to quickly generate results for different scenarios, such as in under a minute, under half a minute, or less.

With the preceding in mind, in certain embodiments, user interaction with the modeling tool is provided via a graphical user interface (GUI) displayed on a general or special purpose computer having processor, memory, storage, display and input components. In such implementations, the GUI may allow the user to position room features (including HVAC features) and imaging system components using a drag-and-drop interface and a mouse or other input structure. Once the room layout is completed (block 86 of FIG. 4), the user may initiate the calculation operations by pushing or selecting a button or other GUI feature. Once initiated the GUI collects all inputs provided by the user, initiates a flow solver, receives the generated air velocity and temperature field results from the flow solver, and displays the results via the GUI. The displayed results, in one embodiment, summarize and represent the temperature and air velocity information in a tabular or graphical form. Alternatively, in certain implementations the GUI may display contour plot of temperatures and air flow velocity, either with or without flow vectors. In certain embodiments, the displayed results also include an estimate or index of patient thermal comfort based on the existing model parameters.

In certain embodiments, the GUI is configured to provide warnings (e.g., visual or audible indicators) for air velocity, temperature, and/or patient comfort values outside a specified range. For example, air temperatures above a desired threshold (or air flow velocities below a certain threshold) at the air inlets or outlets of the modeled imaging system may be visually indicated on the GUI, indicating that the modeled layout is likely not optimal. Similarly, air temperatures below a desired threshold (or air flow velocities above a certain threshold) at the patient table may also indicate that the modeled layout is not optimal from a patient comfort standpoint. In either instance, the user may be prompted to adjust the modeled layout and run the modeling process again. Once a modeling run is completed, if the user is satisfied with the modeled layout, the scan room 50 may be configured in accordance with the satisfactory model, i.e., the scan room HVAC may be configured in accordance with the model and the imaging system components positioned in accordance with the model.

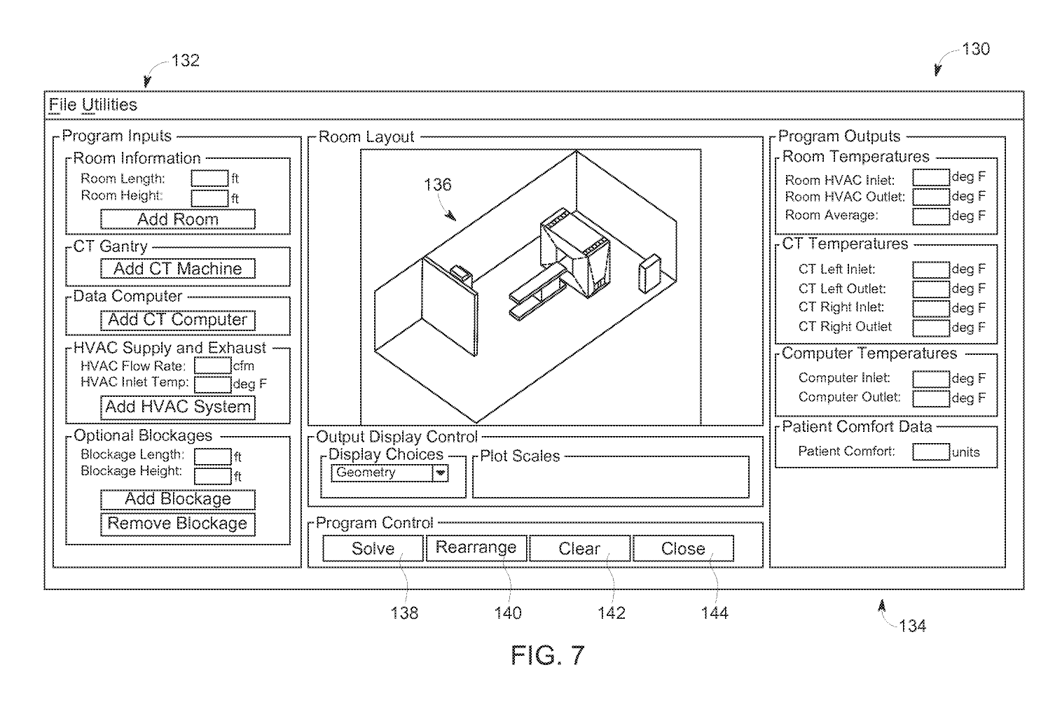

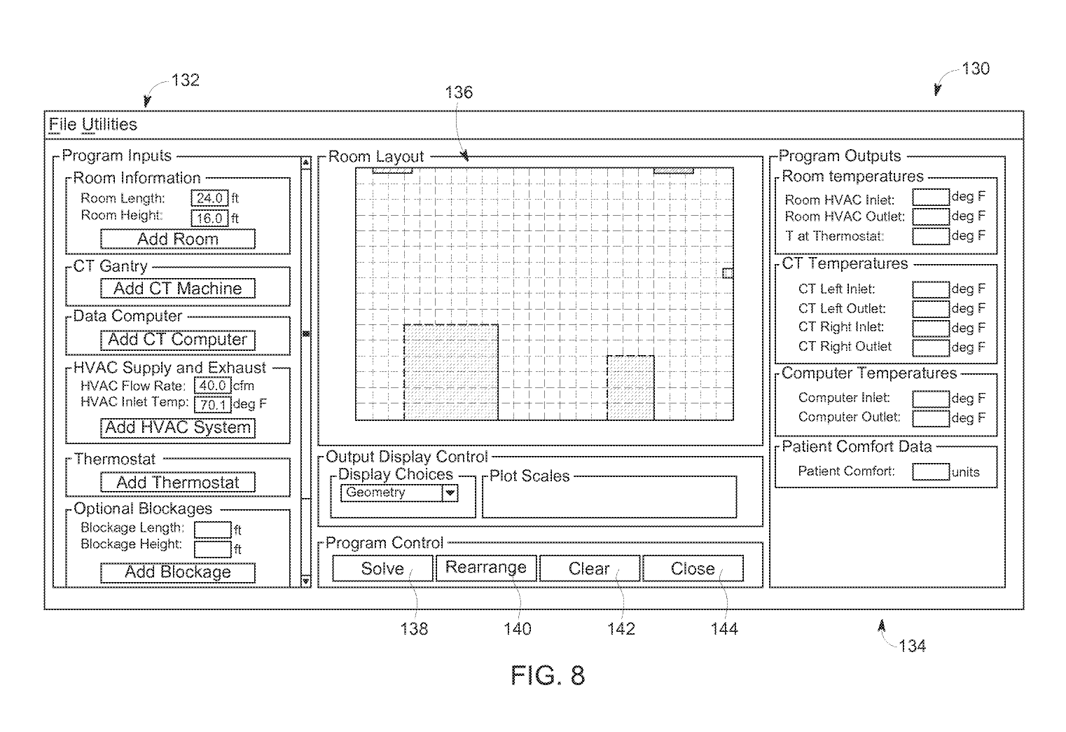

Examples of screens from such a GUI interface are shown as FIGS. 7-10. Turning to FIG. 7, an example of a screen 130 used to input a scan room layout and imaging system configuration is shown. In this example, a left panel 132 provides fields and selectable buttons for providing room dimensions, adding a room, adding an imaging system and computer, inputting HVAC flow rate and temperature, and configuring additional room structures or blockages. Conversely, a right panel 134 displays output fields to be populated on completion of the model run to convey the estimated room temperatures (such as average or median room temperature or temperature at specific, defined locations), estimated imaging system and computer temperatures (such as at the respective inlets and outlets of the imaging system components), and a patient comfort metric. A layout pane 136 provides an area where a user may drag-and-drop visual representations of the room features and imaging system components. That is, within the layout panel 136 the user may define the system and room layout for which the computer mesh will be constructed and which will be modeled in a given run of the tool. Along the bottom of the depicted example screen 130, various button are depicted which allow a user to solve (button 138) the presently entered layout, to rearrange (button 140) the presently entered layout, to clear (button 142) the presently entered layout, and to close (button 144) the tool. Turning to FIG. 8, a similar implementation of an input screen 130 is depicted. Unlike the screen shown in FIG. 7, however, FIG. 8 demonstrates the use of an overhead plan view in the layout pane 136, instead of a perspective or isometric view.

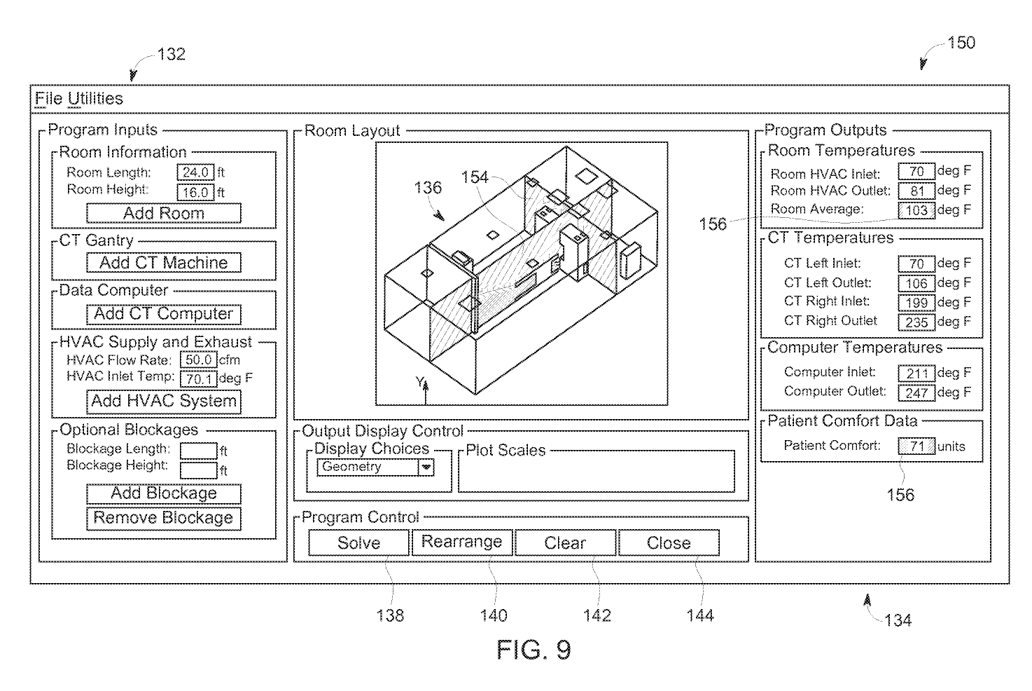

Turning to FIG. 9, an example of an output screen 150 of the GUI is depicted. In this example, the room layout in the layout panel 136 is shown with three-dimensional graphical representations of the estimated temperature profile for the modeled layout. In the depicted example, the temperature profile is show using color or gray-scale shading along representative planes 154 taken through the modeled scan room. Based on these graphical representations a user can see determine the expected temperature at various locations in the modeled scan room. In addition, in the depicted example, the output fields within the output panel 134 are filled in with the calculated values for the respective output fields. In this example, output values that are outside acceptable tolerances are highlighted (shading 156) to bring them to the attention of a reviewer.

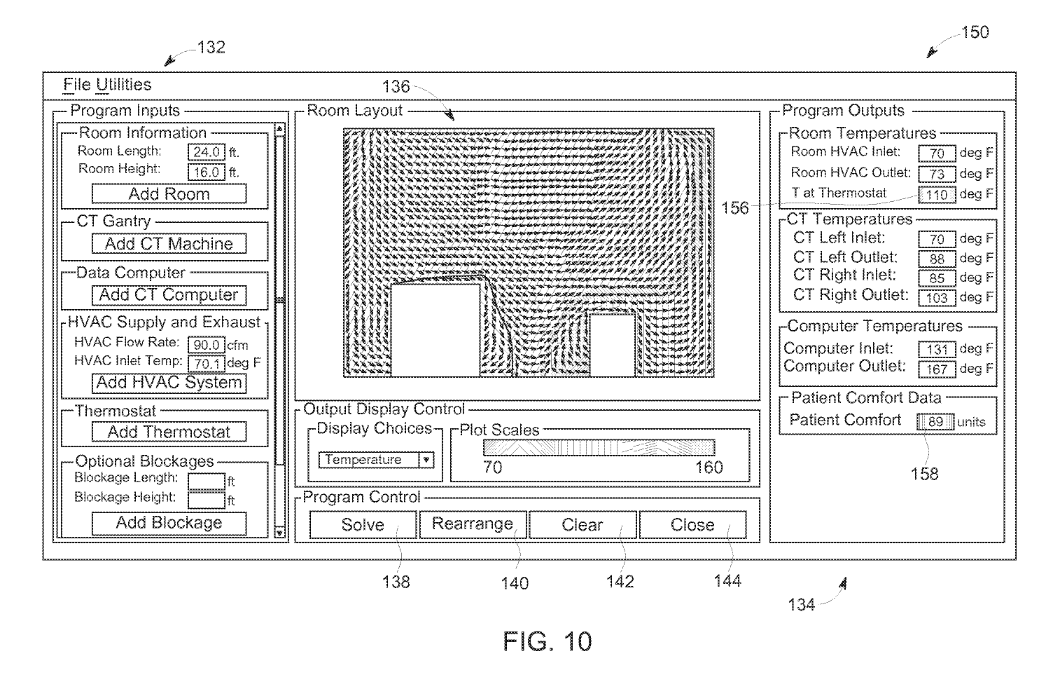

Turning to FIG. 10, a similar implementation of an output screen 150 is depicted. Unlike the screen shown in FIG. 9, however, FIG. 10 demonstrates the use of an overhead plan view in the layout pane 136, instead of a perspective or isometric view. Further, FIG. 10 depicts the temperature profile of the room layout using color or gray-scale and, in addition, is shown depicting air-flow direction using respective arrows or vectors within each modeled cell along the plane shown through the modeled room. In addition, in the depicted example, a different color shading 158 is shown to indicate a value that is within a positive model threshold, i.e., a good or acceptable value.

With the preceding discussion in mind, technical effects of the invention include a computer-implemented tool for evaluating the thermal layout of a scan room in which an imaging system is deployed. In practice, the tool may be used to quickly generate and test different room and imaging system layouts to identify a suitable layout. A scan room and imaging system may then be placed and oriented in accordance with the layout that has been tested and found acceptable using the tool. Another technical effect of the tool is the ability to model each layout using a reduced-physics model in a short time (e.g., under a minute). An additional technical effect is the ability to generate an estimate of patient comfort for each layout tested.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computer-implemented method for modeling patient comfort in a scan room, comprising:
receiving, on a processor-based system, a set of inputs comprising: dimensions and shape of the scan room, placement of one or more imaging system components in the scan room, and placement and operational characteristics of air supplies within the scan room;
generating a cellular mesh based on the set of inputs;
computing an air flow vector for each cell of the cellular mesh based on the set of inputs and a set of boundary conditions;
computing a temperature for each cell of the cellular mesh using the computed air flow vectors and a set of boundary conditions;
computing a measure of projected patient comfort based at least on the computed temperatures, velocities, and the set of inputs; and
displaying the measure of projected patient comfort as a factor in evaluating layout of the scan room described by the set of inputs.

2. The computer-implemented method of claim 1, wherein the operational characteristics of the air supplies specified in the set of inputs comprises a temperature of air exiting the air supplies and a flow rate of air exiting the air supplies.

3. The computer-implemented method of claim 1, further comprising:
after computing the temperature for each cell of the cellular mesh, determining whether one or more boundary temperatures are converged;
if the one or more boundary temperatures are determined to be converged, proceeding to the step of computing the measure of projected patient comfort; and
if the one or more boundary temperatures are not determined to be converged, updating the set of boundary temperatures until the boundary temperatures are determined to be converged.

4. The computer-implemented method of claim 1, wherein the air flow vector for each cell of the cellular mesh is calculated using potential flow theory.

5. The computer-implemented method of claim 1, wherein the air flow vector for each cell of the cellular mesh is calculated using a reduced physics model.

6. The computer-implemented method of claim 1, wherein the temperature for each cell of the cellular mesh is calculated using an energy balancing algorithm.

7. The computer-implemented method of claim 1, wherein the cellular mesh is a hexahedral mesh.

8. The computer-implemented method of claim 1, wherein displaying the measure of projected patient comfort comprises displaying one of a numeric index of patient comfort or a graphical representation depicting an estimated temperature for one or more patient regions.

9. A computer-implemented method for modeling patient comfort in a scan room, comprising:
receiving, on a processor-based system, a set of inputs comprising dimensions and shape of the scan room, placement of one or more imaging system components in the scan room, and placement and operational characteristics of air supplies within the scan room;
generating a cellular mesh based on the set of inputs;
computing an air flow vector for each cell of the cellular mesh based on the set of inputs and a set of boundary conditions;
computing a temperature for each cell of the cellular mesh using the computed air flow vectors;
determining whether one or more boundary temperatures are converged;
if the one or more boundary temperatures are determined to not be converged, updating the set of boundary temperatures until the boundary temperatures are determined to be converged; and
displaying a set of outputs comprising one or more of estimated temperatures of all or part of the scan room or estimated temperatures of all or part of the imaging system components.

10. The computer-implemented method of claim 9, further comprising:
computing a patient comfort estimate based at least on the computed temperatures; and
displaying the patient comfort estimate as one of the set of outputs.

11. The computer-implemented method of claim 10, further comprising visually emphasizing the patient comfort estimate if the patient comfort estimate is above or below specified thresholds.

12. The computer-implemented method of claim 9, wherein the set of outputs comprises an estimated average room temperature or estimated temperatures at one or more of an HVAC inlet or an HVAC outlet.

13. The computer-implemented method of claim 9, wherein the set of outputs comprises an estimated temperature at one or more of an imaging system air inlet or an imaging system air outlet.

14. The computer-implemented method of claim 9, wherein displaying the set of outputs comprises visually emphasizing one or more of the estimated temperatures determined to be above or below specified temperature criteria for the imaging system components.

15. A graphical user interface comprising:
a plurality of input fields, each field corresponding to a parameter defining a patient procedure room or a system to be deployed in the patient procedure room;
a layout pane configured to allow drag-and-drop placement and orientation of the system within the patient procedure room;
a plurality of room temperature fields, each room temperature field corresponding to estimated temperature for all or a portion of the patient procedure room;
a plurality of system temperature fields, each system temperature field corresponding to estimated temperature for a system component; and
one or more patient comfort index fields, each patient comfort index field corresponding to an estimated patient comfort.

16. The graphical user interface of claim 15, wherein the plurality of input fields correspond to parameters comprising one or more of: patient procedure room dimensions, air supply flow rate, and air supply temperature.

17. The graphical user interface of claim 15, further comprising a first user selectable feature configured to cause a model to execute based on the current inputs to the plurality of input fields and the layout pane.

18. The graphical user interface of claim 15, further comprising a second user selectable feature configured to allow a user to rearrange the placement and orientation of the system within the layout pane.

19. The graphical user interface of claim 15, wherein the layout pane is further configures to allow drag-and-drop placement of one or more of a thermostat, a temperature sensor, an air supply, or an air return in the patient procedure room.

20. The graphical user interface of claim 15, wherein the graphical user interface is configured to highlight respective room temperature fields, system temperature fields, or patient comfort index fields when values displayed in the respective fields are above or below specified thresholds.
